(19) United States
(12) Patent Application Publication
(10) Pub. No.: US 2010/0105945 A1
(43) Pub. Date: Apr. 29, 2010

(54) NOBLE METAL CATALYSTS

(76) Inventors: Dorit Wolf, Oberursel (DE); Thomas Riermeier, Ober-Ramstadt (DE); Karlheinz Drauz, Freigericht (DE); Christoph Weber, Wiesbadeb (DE)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/526,593

(22) PCT Filed: Jan. 28, 2008
(86) PCT No.: PCT/EP2008/050950

§ 371 Date: Aug. 10, 2009

(30) Foreign Application Priority Data
Feb. 14, 2007 (DE) 102007007227.0

(51) Int. Cl. C07C 69/66; B01J 31/02; B01J 27/02; C07C 209/28

(52) U.S. Cl. 560/179; 502/168; 564/398

(57) ABSTRACT

Catalyst systems consisting of supported or unsupported transition metal catalysts which have modifiers on the surface. The modifiers have sulphur-containing functionalities (G₀). In addition, the modifiers may have a spacer (Sp) and a Bronsted-basic, Bronsted-acidic or Lewis-basic functionality (G₁). The catalyst systems may be used for hydrogenation, reductive alkylation and reductive amination.

The invention relates to catalyst systems consisting of supported or unsupported transition metal catalysts whose surface has been modified with defined amounts of organic modifiers, to a process for their preparation and to their use.

Owing to their ease of recycleability and their possible use in continuous processes, heterogeneous catalysts find wide use in the production of base chemicals, chemical intermediates, and fine chemical and pharmaceutical products. Fine chemical and pharmaceutical catalytic processes have a high substrate specificity, i.e. particular functional groups in polyfunctional organic substrates have to be converted. The known heterogeneous catalysts usually lead to a lower selectivity of the catalytic reaction compared to homogeneous catalysts.

It is known that the selectivity with respect to particular functional groups of an organic starting molecule can be improved by modifying heterogeneous catalysts with small amounts of organic or inorganic compounds. This modification of heterogeneous catalysts opens up the possibility of widening the scope of application of a commercial solid catalyst because the chemical structure and the amount of the modifier can be adjusted in a controlled manner to the requirements of a particular chemical reaction.

The compounds which are used to modify the catalyst surface are referred to in the technical literature by different terms, for example, modifier, promoter, additive, regulator, selective catalyst poison or co-catalyst.

The term “modifier” is used hereinafter, though this term should be understood to be entirely synonymous with the other names.

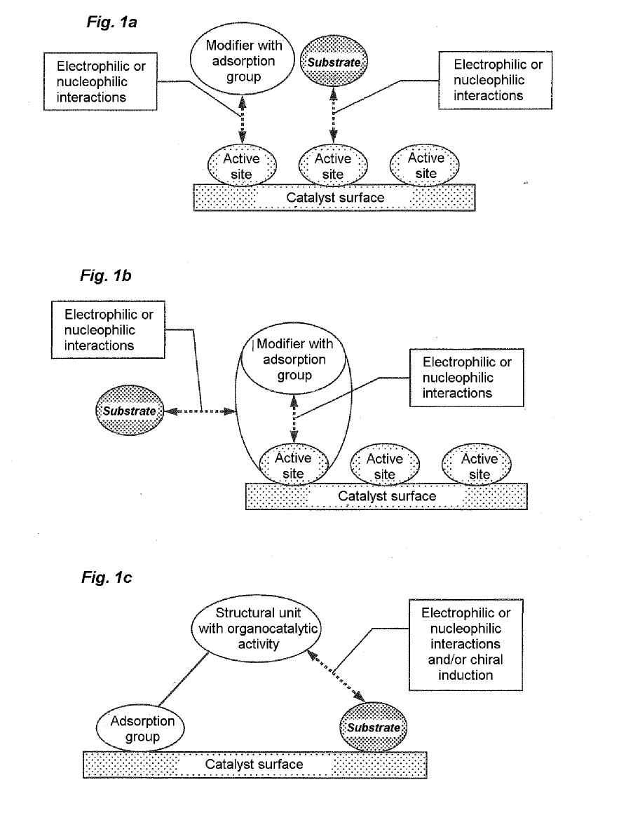

The modifiers have the property of entering into adsorptive interactions with the catalyst surface and in this way inducing desired changes in the activity and selectivity of the catalysts
a) by the variation of the number of active sites on the catalyst surface or
b) by the change in the electronic properties of the active sites on the catalyst surface or
c) by the introduction of organocatalytic functionalities, i.e. by the use of small, simple, possible chiral organic molecules, which can catalyse various reactions in a highly selective manner even without the presence of metals (FIG. 1).

Modifiers for heterogeneous catalysts consist of a structural unit which enables the adhesion (adsorption) of the modifier on the catalyst surface.

In addition, the modifiers for case c) (cf. FIG. 1c) may have structural units with organocatalytic activity. The structural units in question may, for example, be amino acid or peptide structures or organo-metallic complex ligands which, even without the presence of a further metal, can catalyse chemical reactions in a highly selective mannerⁱ.

The organocatalytic functional groups may also have chiral centres, such that the interaction between modifier and reaction substrate can cause chiral induction on the part of the substrates.

The known examples of a change in number or the properties of active sites of the catalyst with modifiers (partial poisoning of the active sites) includes the partial hydrogenation of alkynes to alkenes, in which the most frequently used modifiers are quinoline, but also diamines. This catalyst system finds use in the form of the so-called Lindlar catalystsⁱⁱ. It is assumed that there is competing adsorption of the substrate, of the product and of the modifier.

Addition of nitrogen bases to Pd/C catalysts allows the hydrogenolysis of benzyl ether to be suppressed selectively in the presence of other reducible functional groups such as olefin, benzyl ester, nitro groupsⁱⁱⁱ. However, aromatic N-Cbz (benzyloxycarbonyl) and haloaromatic groups are hydrogenated. In the absence of the N-bases there is in each case complete hydrogenolysis^{iv}.

The use of diphenyl sulphide as a catalyst poison leads to a further expansion of the scope of application of the Pd/C catalyst. For instance, it was possible with a catalyst system modified in this way to hydrogenate olefin and acetylene groups while simultaneously suppressing the hydrogenolysis of aromatic carbonyl and halogen, benzyl ester and N-Cbz groups^{v}. Further S-containing modifiers studied were thiophenol, diphenyl sulphone, diphenyl sulphoxide and diphenyl disulphide.

The examples mentioned for the modification of heterogeneous catalysts have the aim of influencing the chemoselectivity via partial poisoning of the surface. The known modification of heterogeneous catalysts with organic molecules is preparatively simple and inexpensive. Especially in catalytic applications in which the number or properties of the active sites according to FIG. 1a) and b) are influenced by adsorption of simple nitrogen-containing bases and sulphur compounds, many successful catalyst systems are known.

However, when the objective of the catalyst modification is to control stereo-, diastereo- and enantioselectivities, a simple molecule which is adsorbed selectively on the catalyst surface is inadequate.

In this case, the modifier molecules, as well as groups which enable the adsorption on the catalyst surface, require additional organocatalytic functionalities which enter into controlled interactions with the functional groups of the reaction substrate at the surface of the catalyst.

In stereo-, diastereo- and enantioselective reactions in which catalysts having organocatalytic functionalities according to FIG. 1c) are required, the number of successful applications for modified catalysts is still very limited.

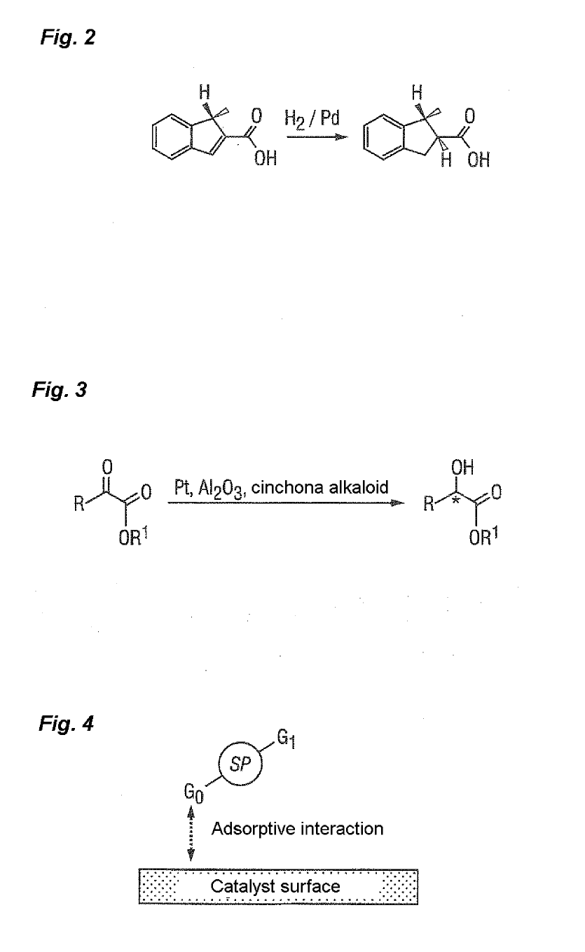

The significance of amines for this type of reaction becomes clear with regard to the hydrogenation of 1-methylindene-2-carboxylic acid (1-MICA) in the presence of Pd/Al₂O₃^{vi} (FIG. 2).

The syn addition of two hydrogen atoms adsorbed on the Pd surface predominantly gives rise to the cis product.

In the case of addition of modifiers (cinchonidine and quinuclidine), the trans/cis ratio is more than doubled. The influence of the tertiary amine modifiers is explained by the acid-base interactions between 1-MICA and the modifier which promotes the adsorption and hydrogenation of 1-MICA in the “upside-down” position.

In the case of enantioselective catalytic reactions, noble metal supported catalysts combined with chiral modifiers can transmit chiral information directly to particular substrate groups.

The combination of Pt/Al₂O₃/cinchona alkaloid allows α-ketocarboxylic esters to be hydrogenated with enantioselectivities of 85-98%^{vi} (FIG. 3).

The stereoselective hydrogenation of β-ketocarboxylic esters^{viii}, with Raney nickel as a catalyst and tartaric acid as a chiral modifier and NaBr as a promoter leads to stereoselectivities for the hydroxyl esters of approximately 80-98%. Further suitable substrates are other β-functionalized ketones and sterically demanding methyl ketones^{ix}.

The combination of palladium with unsubstituted cinchona alkaloids or some vinca alkaloids gives rise to enantioselective catalysts for α,β-unsaturated carboxylic acids (ee up to 74%) and hydroxymethylpyrones (ee up to 94%)^{x}.

Some other supported Pd catalysts with chiral modifiers (for example, amino alcohols, amino acids) have been reported, but the enantioselectivities achieved were only approximately 20-25%.

The overall impression is that the successful applications in the field of stereo-, diastereo- and enantioselective reactions are restricted to readily activable substrates which are converted under mild reaction conditions (low H₂ pressure in the case of hydrogenation, low temperature).

One cause of this is suspected to lie in the limited inertness and in the undesirable degradation of the chiral modifier during the catalytic reaction.

For instance, it is known that cinchona modifiers which are used in the enantioselective hydrogenation in conjunction with Pt catalysts are adsorbed as a result of the interaction between their aromatic ring system and the catalyst surface. This aromatic group is, however, hydrogenated during the reaction. This leads to the detachment of the modifier from the catalyst and hence to the decline or complete loss of selectivity.

Furthermore, adsorption groups which enter into more labile adsorption interactions have the disadvantage that the adsorption of these molecules requires specific metal surfaces or adsorption sites. The usability of corresponding modifiers is therefore tied to particular metal particle structures, support materials and to narrowly-specified preparation methods of the heterogeneous catalysts.

Functioning enantioselective Pt-cinchona alkaloid systems are based, for example, on Al₂O₃ as the support material. Activated carbon-supported catalysts, in contrast, exhibit only low selectivities.

It is an object of the invention, therefore, to develop catalyst systems with robust organic modifiers which have both organocatalytic functionalities and adsorption groups which enable strong unspecific adsorption on the catalyst surface. These inventive catalyst systems can activate comparatively unreactive substrates under relatively severe reaction conditions (elevated temperature, elevated pressure) and convert them chemo-, stereo-, diastereo- and/or enantioselectively.

The invention provides catalyst systems consisting of supported or unsupported transition metal catalysts whose surface has been modified with defined amounts of organic modifiers, which are characterized in that the modifier has a sulphur-containing functionality (G₀).

Even though, according to the prior art, sulphur-containing molecules are known predominantly for the poisoning of catalysts, it has been found in the case of the inventive catalysts which are treated with sulphur compounds that, surprisingly, an increase both in activity and selectivity can occur compared to unmodified catalysts.

The inventive catalyst system may consist of an unsupported catalyst or a supported catalyst and an organic modifier and be characterized in that the modifier has, as a sulphur-containing functionality (Gₒ) thiol, (poly)sulphane, thiophene or thiopyran groups.

The inventive catalyst system may be characterized in that the modifier has at least one further functional group (G₁) with Brønsted-basic, Brønsted-acidic, Lewis-basic or Lewis-acidic properties.

The inventive catalyst system may be characterized in that the modifier has a spacer (Sp) between the sulphur-containing functionality (G₀) and the Brønsted-basic, Brønsted-acidic or Lewis-basic functionality (G₁).

The inventive catalyst system may be characterized in that the unsupported catalyst or the supported catalyst comprises one or more catalytically active components, where these components may be compounds of the elements of transition group I, II, VII and VIII of the Periodic Table and preferably compounds of the elements Pt, Pd, Rh, Ru, Re, Ir, Au, Ag, Ni, Co, Cu and Fe.

The inventive catalyst system may be characterized in that the modifier is adsorbed on the catalyst surface during or immediately after the preparation of the metal or supported metal catalyst and is introduced into the catalytic process stage as such a catalyst system.

The inventive catalyst system may be characterized in that the modifier is adsorbed on the catalyst surface immediately before the introduction into the catalytic process stage.

The inventive catalyst system may be characterized in that the modifier and the heterogeneous catalyst are introduced into the catalytic process stage, and the modifier is adsorbed on the catalyst surface in situ.

The inventive catalyst system may be characterized in that the modifier, as a sulphur-containing functionality (G₀) has alkylthiol or alkylsulphane or alkyldisulphane or alkyltrisulphane or alkylpolysulphane groups, or arylthiol or arylsulphane or aryldisulphane or aryltrisulphane or arylpolysulphane groups, or alkylarylthiol or alkylarylsulphane or alkylaryldisulphane or alkylalkyltrisulphane or alkylarylpolysulphane groups.

The inventive catalyst system may be characterized in that the modifier preferably has, as a sulphur-containing functionality (G₀), phenylthiol or phenylsulphane groups or benzylthiol or benzylsulphane groups.

The inventive catalyst system may be characterized in that the mass ratio of modifier:catalyst is in the range between 10 000:1 and 1:10 000 and preferably between 10:1 and 1:1000.

The inventive catalyst system may be characterized in that the modifier has, as a functional group (G₁) one or more groups from the group of

amino and/or
carboxylic acid and/or
carboxylic ester and/or
carboxamide and/or
aminocarboxylic acid and/or
aminocarboxylic ester and/or
aminocarboxamide and/or
hydroxycarboxylic acid and/or
hydroxycarboxylic ester and/or
hydroxycarboxamide and/or
aminoalcohol and/or
diol and/or
urea and/or
thiourea.

Preferred modifiers with a sulphur-containing functionality (Gₒ) according to the invention may be organic molecules which contain thiol, (poly)sulphane, thiophene or thiopyran groups and additionally also have at least one further functional group (G₁) with Brønsted-basic, Brønsted-acidic, or Lewis-basic properties, for example amino, amino acid, hydroxycarboxylic acid, aminoalcohol, diol, biphenol, urea or thiourea groups.

The modifiers of the inventive catalysts may have a spacer (Sp) which is disposed between functionality G₀ and G₁. The spacer may have, for example, the structures detailed in Table 1.

Examples of such modifiers are compiled in FIG. 4 and Table 1.

TABLE 1
Examples of the functional groups Sp, G₀ and G₁ of the
inventive modifiers

| Sp | G₀ | G₁ |
|---|---|---|
| 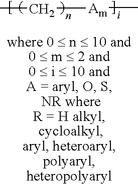 | 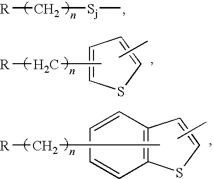 | 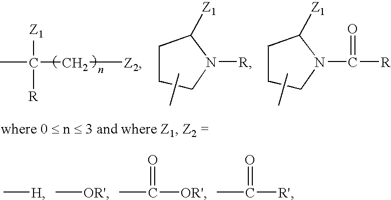 |
| | 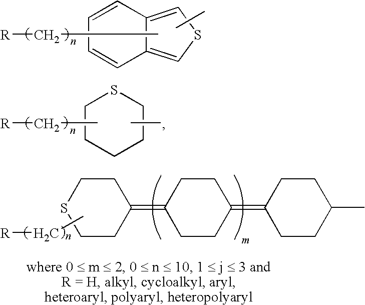 | 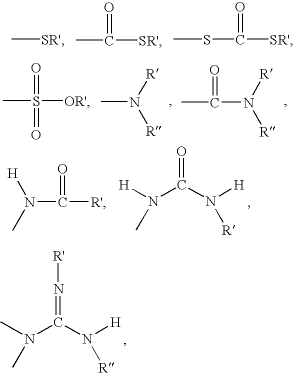 |
| | | 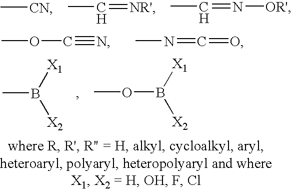 |

The S-containing functionalities Gₒ of the modifiers of the inventive catalyst system documented in FIG. 4 can serve for the strong adsorption of the modifier on the metal surface, which is maintained even in the case of elevated reaction temperature and high concentrations of reactive substrates.

The modifiers of the inventive catalysts may have at least one chiral centre.

The inventive catalyst system may be characterized in that the catalyst system can catalyse reactions of the following reaction classes:

chemo-, stereo-, diastereo- and/or enantioselective hydrogenations of substrates which contain
one or more carbonyl groups and/or
one or more C═C double bonds and/or
one or more aromatic and/or heteroaromatic groups and/or
one or more nitro groups and/or
one or more nitrile groups and/or
one or more imine groups and/or
one or more hydroxylamine groups and/or
one or more alkyne groups,

the chemo-, stereo-, diastereo-, or enantioselective reductive alkylation of primary or secondary amines or

the chemo-, stereo-, diastereo- or enantioselective reductive amination of aldehydes or ketones with ammonium salts or amines.

The temperature range of the catalytic use of the inventive catalysts may be −70 to 220° C., preferably −10 to 200° C. and especially 20 to 140° C.

The pressure range (partial H₂ pressure) of the catalytic use of the inventive catalysts may be 0.1 to 300 bar, preferably 0.5 to 100 bar.

The mass ratio of catalyst:modifier of the inventive catalyst may be between 1:1 and 10 000:1, preferably between 10:1 and 1000:1.

With the varying functionalities Z₁ and Z₂ of the group G₁ (see Table 1 and FIG. 4), it is possible to control the chemo-, stereo-, diastereo- and/or enantio-selectivity of the catalytic reaction of different reaction and substrate classes.

The inventive catalyst system can be used to catalyse the following reaction classes:

chemo-, stereo-, diastereo- and/or enantioselective hydrogenation of substrates which have at least one functional group or a plurality of functional groups from the group of:
one or more carbonyl groups,
one or more C═C double bonds,
one or more aromatic and/or heteroaromatic groups,
one or more nitro groups,
one or more nitrile groups,
one or more imine groups,
one or more hydroxylamine groups,
one or more alkyne groups.

The inventive catalyst system can also be used for the chemo-, stereo-, diastereo- or enantioselective reductive alkylation of primary or secondary amines.

The inventive catalyst system can also be used for the chemo-, stereo-, diastereo- or enantioselective reductive amination of aldehydes or ketones with ammonium salts or amines.

The active metal components of the inventive catalyst system may consist of one or more noble metals such as Pd, Pt, Ag, Au, Rh, Ru, Ir, and/or further transition metals such as Ni, Cu, Co, Mo.

The catalysts may comprise further elements, for example, alkali metals and alkaline earth metals, elements of main group 3, 4 and 5 and/or elements of transition group 1 to 8.

The metal components of the catalysts may be applied to supports, in which case the supports used may be activated carbons, carbon black and oxidic materials such as Al₂O₃, SiO₂, TiO₂, ZrO₂, aluminosilicates, MgO, CaO, SrO, BaO, or mixed oxides composed of the oxides mentioned.

The novel inventive robust organic modifiers allow effective modification of different supported metal catalysts and are no longer restricted to narrowly specified support and metal particle properties.

The resulting inventive catalyst systems open up access to a multitude of chemo-, stereo-, diastereo- and enantioselective chemical reactions.

### EXAMPLES

The examples concentrate on the use of inventive modified catalysts in reactions in which elevated reaction temperatures and partial hydrogen pressures are required for the substrate activation and for which the inventive catalyst systems have a significant improvement compared to the prior art.

### Example 1

### Heterogeneously Catalysed Enantioselective Reductive Amination in the Presence of Pt Catalysts which have been Modified with Amino Acid Sulphane/Thiol Derivatives

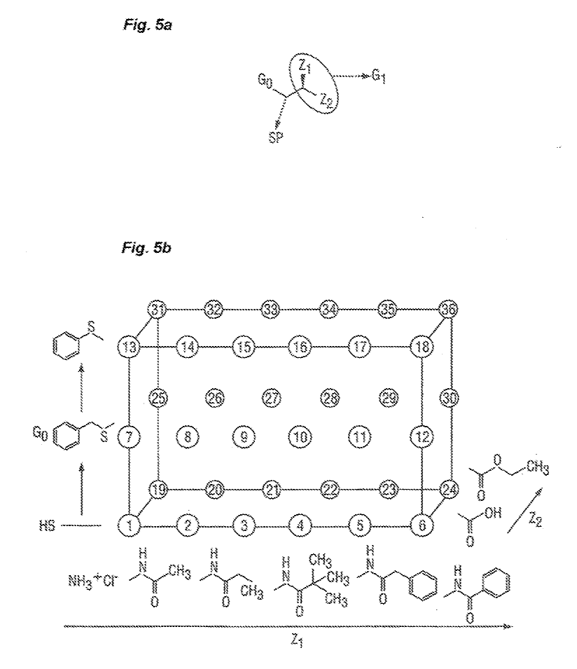

A library of 36 modifiers was generated. This library is based on the α-amino acid base structure shown in FIG. 5a. The substituents G₀, G₁ and, within the group G₁ the functionalities Z₁ and Z₂ (see also Table 1) were varied systematically according to FIG. 5b.

The representatives of the substance library according to FIG. 5 were used for the modification of different Pt catalysts. These catalysts each contained 5% by mass of Pt on an Al₂O₃ support (corresponds to Catasium F214 in Table 1a and b) or 3% by mass of Pt on an activated carbon support (corresponds to F1082QHA/W3% in Table 1a and b). The modified Pt catalysts were used in the reductive amination of ethyl phenyl ketone to propylphenylamine.

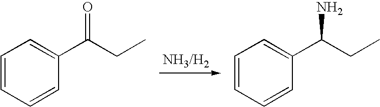

The reaction was performed in a pressure reactor at a partial H₂ pressure of 30 bar and a reaction temperature of 50° C. to 80° C. in methanol as a solvent. The catalysts were suspended in 3 ml of the solvent. Thereafter, 1 ml of the solution of the modifier in the solvent was added and the mixture was stirred at room temperature for 30 min. Thereafter, 1 ml of the substrate solution and 1 ml of the solution of the ammonium salt were added. The reactor was first purged with nitrogen and then charged with hydrogen up to the intended reaction pressure, and the reaction temperature was established. At the start of the reaction, the molar ethyl phenyl ketone:NH₄OH ratio was 1:3. The molar ratio of substrate to modifier was varied in the range of 1:1 to 10 000:1. Table 2a) and b) contain yields or propylphenylamine and ee values for selected experiments of these variations. It is found that, especially with the inventive catalyst/modifier systems No. 8, 11, 12, 14, 15, 16, 17, 18, 29, 30, 32, 35, 36 (Table 2a, b), enantio-selectivities are achieved which are both above the ee values of a sulphur-free modifier analogue (N-acetylphenylalanine), and above the ee values which are obtained without use of a modifier.

TABLE 2a

| Number | | | | | | |
|---|---|---|---|---|---|---|
| of the | | | Mass | | | |
| modifier | | | of | | | Reaction |
| (see | | | catalyst/ | Temp/ | | time/ |
| FIG. 6b) | Modifier | Catalyst | mg | ° C. | p/bar | min |
| 7 | S-benzyl-L-cysteine*HCl | Catasium F218 | 30 | 56 | 30 | 1028 |
| 7 | S-benzyl-L-cysteine*HCl | F 1082 QHA/W 3% | 30 | 57 | 30 | 1028 |
| 8 | N—Ac—S-benzyl-L-cysteine | F 1082 QHA/W 3% | 30 | 58 | 30 | 1028 |
| 8 | N—Ac—S-benzyl-L-cysteine | F 1082 QHA/W 3% | 30 | 57 | 30 | 1070 |
| 8 | N—Ac—S-benzyl-L-cysteine | F 1082 QHA/W 3% | 30 | 57 | 30 | 1028 |
| 8 | N—Ac—S-benzyl-L-cysteine | F 1082 QHA/W 3% | 30 | 57 | 30 | 1028 |
| 8 | N—Ac—S-benzyl-L-cysteine | F 1082 QHA/W 3% | 30 | 56 | 30 | 1028 |
| 8 | N—Ac—S-benzyl-L-cysteine | F 1082 QHA/W 3% | 30 | 55 | 30 | 1028 |
| 8 | N—Ac—S-benzyl-L-cysteine | F 1082 QHA/W 3% | 30 | 55 | 30 | 1028 |
| 9 | N-propionyl-S-benzyl-L- | Catasium F214 | 30 | 55 | 30 | 1020 |
| | cysteine | | | | | |
| 9 | N-propionyl-S-benzyl-L- | F 1082 QHA/W 3% | 30 | 55 | 30 | 1020 |
| | cysteine | | | | | |
| 10 | N-trimethylacetyl-S- | Catasium F214 | 30 | 54 | 30 | 1020 |
| | benzyl-L-cysteine | | | | | |
| 10 | N-trimethylacetyl-S- | F 1082 QHA/W 3% | 30 | 55 | 30 | 1020 |
| | benzyl-L-cysteine | | | | | |
| 11 | N-benzyl-S-benzyl-L- | Catasium F214 | 30 | 55 | 30 | 1020 |
| | cysteine | | | | | |
| 11 | N-benzyl-S-benzyl-L- | F 1082 QHA/W 3% | 30 | 54 | 30 | 1020 |
| | cysteine | | | | | |
| 12 | N-phenylacetyl-S-benzyl- | Catasium F214 | 30 | 55 | 30 | 1020 |
| | L-cysteine | | | | | |
| 12 | N-phenylacetyl-S-benzyl- | F 1082 QHA/W 3% | 30 | 55 | 30 | 1020 |
| | L-cysteine | | | | | |
| 13 | S-phenyl-L-cysteine*HCl | Catasium F214 | 30 | 55 | 30 | 1070 |
| 13 | S-phenyl-L-cysteine*HCl | F 1082 QHA/W 3% | 30 | 55 | 30 | 1070 |
| 14 | N—Ac—S-phenyl-L-cysteine | Catasium F214 | 30 | 55 | 30 | 1070 |
| 14 | N—Ac—S-phenyl-L-cysteine | F 1082 QHA/W 3% | 30 | 55 | 30 | 1070 |
| 15 | N-propionyl-S-phenyl-L- | Catasium F214 | 30 | 55 | 30 | 1070 |
| | cysteine | | | | | |
| 15 | N-propionyl-S-phenyl-L- | F 1082 QHA/W 3% | 30 | 55 | 30 | 1070 |
| | cysteine | | | | | |
| 16 | N-trimethylacetyl-S- | Catasium F214 | 30 | 55 | 30 | 1070 |
| | phenyl-L-cysteine ethyl | | | | | |
| 16 | N-trimethylacetyl-S- | F 1082 QHA/W 3% | 30 | 55 | 30 | 1070 |
| | phenyl-L-cysteine ethyl | | | | | |
| 17 | N-benzyl-S-phenyl-L- | Catasium F214 | 30 | 55 | 30 | 1012 |
| | cysteine | | | | | |
| 17 | N-benzyl-S-phenyl-L- | F 1082 QHA/W 3% | 30 | 55 | 30 | 1012 |
| | cysteine | | | | | |
| 18 | N-phenylacetyl-S-phenyl- | Catasium F214 | 30 | 55 | 30 | 1012 |
| | L-cysteine | | | | | |
| 18 | N-phenylacetyl-S-phenyl- | F 1082 QHA/W 3% | 30 | 55 | 30 | 1012 |
| | L-cysteine | | | | | |

| Number | | | | | |
|---|---|---|---|---|---|
| of the | | | | | |
| modifier | | | | | |
| (see | c(Substrate)/ | n(NH₄OH)/ | n(subs.)/ | Ketone | Amine |
| FIG. 6b) | g/l | n(subs.) | n(mod.) | conversion/% | yield/% |
| 7 | 0.1 | 3.0 | 100 | 28 | 28 |
| 7 | 0.1 | 3.0 | 100 | 27 | 27 |
| 8 | 0.1 | 2.9 | 5 | 33 | 33 |
| 8 | 0.1 | 2.8 | 11 | 30 | 27 |
| 8 | 0.1 | 3.0 | 52 | 28 | 26 |
| 8 | 0.1 | 3.0 | 54 | 31 | 29 |
| 8 | 0.1 | 3.0 | 106 | 30 | 23 |
| 8 | 0.1 | 2.9 | 107 | 29 | 23 |
| 8 | 0.1 | 2.9 | 500 | 34 | 30 |
| 9 | 0.1 | 3.0 | 100 | 22 | 17 |
| 9 | 0.1 | 3.0 | 100 | 22 | 18 |
| 10 | 0.1 | 3.0 | 100 | 10 | 10 |
| 10 | 0.1 | 3.0 | 100 | 12 | 12 |
| 11 | 0.1 | 3.0 | 100 | 21 | 20 |
| 11 | 0.1 | 3.0 | 100 | 23 | 23 |
| 12 | 0.1 | 3.0 | 100 | 17 | 17 |
| 12 | 0.1 | 3.0 | 100 | 21 | 20 |
| 13 | 0.1 | 3.0 | 100 | 19 | 28 |
| 13 | 0.1 | 3.0 | 100 | 21 | 27 |
| 14 | 0.1 | 3.0 | 100 | 32 | 31 |
| 14 | 0.1 | 3.0 | 100 | 31 | 30 |
| 15 | 0.1 | 3.0 | 100 | 28 | 19 |
| 15 | 0.1 | 3.0 | 100 | 29 | 21 |
| 16 | 0.1 | 3.0 | 100 | 23 | 18 |
| 16 | 0.1 | 3.0 | 100 | 27 | 20 |
| 17 | 0.1 | 3.0 | 100 | 27 | 26 |
| 17 | 0.1 | 3.0 | 100 | 29 | 28 |
| 18 | 0.1 | 3.0 | 100 | 31 | 30 |
| 18 | 0.1 | 3.0 | 100 | 30 | 28 |

TABLE 2b

| Number of | | | | | | |
|---|---|---|---|---|---|---|
| the | | | Mass | | | |
| modifier | | | of | | | Reaction |
| (see FIG. | | | catalyst/ | Temp/ | | time/ |
| 6b) | Modifier | Catalyst | mg | ° C. | p/bar | min |
| 19 | L-cysteine ethyl | Catasium F 214 | 10 | 55 | 30 | 1046 |
| | ester*HCl | | | | | |
| 21 | N-propionyl-L-cysteine | F 1082 QHA/W 3% | 30 | 57 | 30 | 1048 |
| | ethyl ester | | | | | |
| 21 | N-propionyl-L-cysteine | F 1082 QHA/W 3% | 30 | 56 | 30 | 1048 |
| | ethyl ester | | | | | |
| 24 | N-phenylacetyl-L- | F 1082 QHA/W 3% | 30 | 56 | 31 | 1080 |
| | cysteine ethyl ester | | | | | |
| 25 | S-benzyl-L-cysteine | Catasium F214 | 30 | 57 | 31 | 990 |
| | ethyl ester*HCl | | | | | |
| 25 | S-benzyl-L-cysteine | F 1082 QHA/W 3% | 30 | 54 | 34 | 990 |
| | ethyl ester*HCl | | | | | |
| 26 | N—Ac—S-benzyl-L-cysteine | Catasium F214 | 30 | 55 | 31 | 990 |
| | ethyl ester | | | | | |
| 26 | N—Ac—S-benzyl-L-cysteine | F 1082 QHA/W 3% | 30 | 53 | 31 | 990 |
| | ethyl ester | | | | | |
| 27 | N-propionyl-S-benzyl-L- | Catasium F214 | 30 | 56 | 31 | 990 |
| | cysteine ethyl ester | | | | | |
| 27 | N-propionyl-S-benzyl-L- | F 1082 QHA/W 3% | 30 | 56 | 30 | 990 |
| | cysteine ethyl ester | | | | | |
| 29 | N-benzyl-S-benzyl-L- | Catasium F214 | 30 | 57 | 31 | 1040 |
| | cysteine ethyl ester | | | | | |
| 29 | N-benzyl-S-benzyl-L- | F 1082 QHA/W 3% | 30 | 56 | 31 | 1040 |
| | cysteine ethyl ester | | | | | |
| 30 | N-phenylacetyl-S-benzyl- | Catasium F214 | 30 | 55 | 32 | 1040 |
| | L-cysteine ethyl ester | | | | | |
| 30 | N-phenylacetyl-S-benzyl- | F 1082 QHA/W 3% | 30 | 55 | 30 | 1040 |
| | L-cysteine ethyl ester | | | | | |
| 31 | S-phenyl-L-cysteine | Catasium F214 | 30 | 55 | 30 | 1040 |
| | ethyl ester*HCl | | | | | |
| 31 | S-phenyl-L-cysteine | F 1082 QHA/W 3% | 30 | 54 | 31 | 1040 |
| | ethyl ester*HCl | | | | | |
| 32 | N—Ac—S-phenyl-benzyl-L- | Catasium F214 | 30 | 55 | 30 | 1040 |
| | cysteine ethyl ester | | | | | |
| 32 | N—Ac—S-phenyl-L-cysteine | F 1082 QHA/W 3% | 30 | 54 | 30 | 1040 |
| | ethyl ester | | | | | |
| 33 | N-propionyl-S-phenyl-L- | Catasium F214 | 30 | 56 | 30 | 1040 |
| | cysteine ethyl ester | | | | | |
| 33 | N-propionyl-S-phenyl-L- | F 1082 QHA/W 3% | 30 | 55 | 30 | 1040 |
| | cysteine ethyl ester | | | | | |
| 34 | N-trimethylacetyl-S- | Catasium F214 | 30 | 55 | 30 | 1040 |
| | phenyl-L-cysteine ethyl | | | | | |
| | ester | | | | | |
| 34 | N-trimethylacetyl-S- | F 1082 QHA/W 3% | 30 | 56 | 30 | 1040 |
| | phenyl-L-cysteine ethyl | | | | | |
| | ester | | | | | |
| 35 | N-benzyl-S-phenyl-L- | Catasium F214 | 30 | 55 | 30 | 1040 |
| | cysteine ethyl ester | | | | | |
| 35 | N-benzyl-S-phenyl-L- | F 1082 QHA/W 3% | 30 | 55 | 30 | 1040 |
| | cysteine ethyl ester | | | | | |
| 36 | N-phenylacetyl-S-phenyl- | Catasium F214 | 30 | 54 | 30 | 1040 |
| | L-cysteine ethyl ester | | | | | |
| 36 | N-phenylacetyl-S-phenyl- | F 1082 QHA/W 3% | 30 | 56 | 30 | 1040 |
| | L-cysteine ethyl ester | | | | | |
| Reference | N-acetylphenylalanine | Catasium F214 | 30 | 55 | 30 | 1000 |
| Reference | N-acetylphenylalanine | F 1082 QHA/W 3% | 30 | 55 | 30 | 1000 |
| Reference | No modifier | Catasium F214 | 11 | 55 | 31 | 980 |
| Reference | No modifier | F 1082 QHA/W 3% | 29 | 56 | 31 | 1080 |

| Number of | | | | | |
|---|---|---|---|---|---|
| the | | | | | |
| modifier | | | | | |
| (see FIG. | c(Substrate)/ | n(NH₄OH)/ | n(subs.)/ | Ketone | Amine |
| 6b) | g/l | n(subs.) | n(mod.) | conversion/% | yield/% |
| 19 | 0.1 | 3.6 | 9 | 8 | 8 |
| 21 | 0.1 | 3.1 | 54 | 3 | 3 |
| 21 | 0.1 | 3.0 | 219 | 8 | 8 |
| 24 | 0.1 | 3.1 | 217 | 9 | 9 |
| 25 | 0.1 | 2.8 | 109 | 22 | 22 |
| 25 | 0.1 | 2.8 | 109 | 22 | 22 |
| 26 | 0.1 | 2.8 | 219 | 20 | 20 |
| 26 | 0.1 | 2.8 | 219 | 23 | 23 |
| 27 | 0.1 | 2.8 | 100 | 20 | 20 |
| 27 | 0.1 | 2.8 | 100 | 20 | 20 |
| 29 | 0.1 | 3.0 | 100 | 21 | 21 |
| 29 | 0.1 | 3.0 | 100 | 36 | 35 |
| 30 | 0.1 | 3.0 | 100 | 25 | 25 |
| 30 | 0.1 | 3.0 | 100 | 36 | 36 |
| 31 | 0.1 | 3.0 | 100 | 29 | 27 |
| 31 | 0.1 | 3.0 | 100 | 33 | 31 |
| 32 | 0.1 | 3.0 | 100 | 17 | 16 |
| 32 | 0.1 | 3.0 | 100 | 30 | 29 |
| 33 | 0.1 | 3.0 | 100 | 15 | 15 |
| 33 | 0.1 | 3.0 | 100 | 22 | 22 |
| 34 | 0.1 | 3.0 | 100 | 19 | 19 |
| 34 | 0.1 | 3.0 | 100 | 24 | 24 |
| 35 | 0.1 | 3.0 | 100 | 21 | 20 |
| 35 | 0.1 | 3.0 | 100 | 32 | 31 |
| 36 | 0.1 | 3.0 | 100 | 22 | 21 |
| 36 | 0.1 | 3.0 | 100 | 35 | 34 |
| Reference | 0.1 | 3.0 | 100 | 30 | 16 |
| Reference | 0.1 | 3.0 | 100 | 28 | 16 |
| Reference | 0.1 | 2.8 | 0 | 17 | 17 |
| Reference | 0.1 | 3.0 | 0 | 33 | 30 |

### Example 2

Representative No. 8 of the substance library according to FIG. 5 was used for the modification of a Pt catalyst (5% by mass of Pt supported on Al₂O₃). The catalyst was obtained by suspending 3 g of aluminium oxide at room temperature in 40 ml of 2.5% sodium carbonate solution (Na₂CO₃) with a magnetic stirrer at 50° C. for 15 min. 400 mg of hexachloroplatinic acid hexahydrate (H₂PtCl₆*6H₂O corresponding to 150 mg of Pt), dissolved in 30 ml of water, were added dropwise to the support suspension within approx. 30 min.

After the addition had ended, the mixture was stirred for another 15 min and then the pH was adjusted to 10.5. The reduction was effected by adding 0.3 g of sodium borohydride (NaBH₄) in 30 ml of water at 50° C. After the reduction had set in (recognizable by immediate blackening of the catalyst), the mixture was stirred for another about 45 min, before the catalyst was removed with a frit, washed with water and dried overnight at approx. 70° C. in a drying cabinet.

Immediately after the preparation, the catalyst was suspended in 40 ml of a methanol solution which contained 0.4 mmol/l of modifier No. 8 (cf. FIG. 5). Thereafter, the solid was filtered off again, optionally washed with water and dried at room temperature in a vacuum cabinet.

The modified Pt catalysts were used in the reductive amination of ethyl phenyl ketone to propylphenylamine.

The reaction was performed in a pressure reactor at a partial H₂ pressure of 30 bar and a reaction temperature of 50° C. in methanol as a solvent. The catalyst was suspended in 4 ml of the solvent. Thereafter 1 ml of the substrate solution and 1 ml of the solution of the ammonium salt were added. The reactor was first purged with nitrogen and then charged with hydrogen up to the intended reaction pressure, and the reaction temperature was established. At the start of the reaction, the molar ethyl phenyl ketone:NH₄OH ratio was 1:3.

Table 3 shows yields of propylphenylamine and ee values which are significantly above the values of the unmodified catalyst (cf. Example 1, Table 2b).

TABLE 3

| Number | | | | | | | |
|---|---|---|---|---|---|---|---|
| of the | | | | | | | |
| modifier | | | Mass of | | | Reaction | |
| (see | | | catalyst/ | Temp/ | | time/ | c(Substrate)/ |
| FIG. 6b) | Modifier | Catalyst | mg | ° C. | p/bar | min | g/l |
| 8 | N—Ac—S-benzyl- | Pt/Al₂0₃ | 10.3 | 56 | 30.2 | 1070 | 0.1 |
| | L-cysteine | | | | | | |
| 8 | N—Ac—S-benzyl- | Pt/Al₂O₃ | 10.0 | 55 | 30.3 | 1070 | 0.1 |
| | L-cysteine | | | | | | |

| Number | | | | | | |
|---|---|---|---|---|---|---|
| of the | | | | | | |
| modifier | | | | | | |
| (see | n(NH₄OH)/ | n(subs.)/ | Ketone | Amine | Amine | |
| FIG. 6b) | n(subs.) | n(mod.) | conversion/% | yield/% | selectivity/% | ee |
| 8 | 2.8 | 11 | 21.0 | 21 | 100.0 | 20.8 |
| 8 | 2.8 | 110 | 23.0 | 23 | 100.0 | 26.5 |

### Example 3

Representative No. 8 in the substance library according to FIG. 5 was used for the modification of a Pt catalyst (3% by mass of Pt supported on activated carbon, referred to as F1082QHA/W3%).

The Pt catalyst was used in the reductive amination of ethyl phenyl ketone to propylphenylamine and modified in situ with N-Ac—S-benzyl-L-cysteine.

The reaction was performed in a pressure reactor at a partial H₂ pressure of 30 bar and a reaction temperature of 50° C. to 80° C. in methanol as a solvent. The catalyst was suspended in 3 ml of the solvent. The reactor was first purged with nitrogen and then charged with hydrogen up to the intended reaction pressure, and the reaction temperature was established. Thereafter, 3 ml of a methanol solution which comprised the modifier NH₄OH and the substrate were added to the catalyst suspension under reaction conditions with stirring. The molar ethyl phenyl ketone:NH₄OH ratio was 1:3. The molar substrate:modifier ratio in the reactor was 1:11.

Table 4 shows yields of propylphenylamine and ee values which are significantly above the values of the unmodified catalyst (cf. Example 1, Table 2b).

TABLE 4

| Number | | | | | | |
|---|---|---|---|---|---|---|
| of the | | | Mass | | | |
| modifier | | | of | | | Reaction |
| (see | | | catalyst/ | Temp/ | | time/ |
| FIG. 6b) | Modifier | Catalyst | mg | ° C. | p/bar | min |
| 8 | N—Ac—S-benzyl- | F 1082 QHA/W 3% | 9.9 | 55 | 30.1 | 1070 |
| | L-cysteine | | | | | |
| 8 | N—Ac—S-benzyl- | F 1082 QHA/W 3% | 9.8 | 57 | 30 | 1070 |
| | L-cysteine | | | | | |

| Number | | | | | | |
|---|---|---|---|---|---|---|
| of the | | | | | | |
| modifier | | | | | | |
| (see | c(Substrate)/ | n(NH₄OH)/ | n(subs.)/ | Ketone | Amine | Amine |
| FIG. 6b) | g/l | n(subs.) | n(mod.) | conversion/% | yield/% | selectivity |
| 8 | 0.1 | 2.8 | 11 | 44.2 | 22.9 | 51.9 |
| 8 | 0.1 | 2.8 | 11 | 44.2 | 26.7 | 60.4 |

### Example 4

### Heterogeneously Catalysed Enantioselective Hydrogenations of α-Keto Carboxylic Acid Derivatives

For the enantioselective hydrogenation of ethyl pyruvate, a Pt/Al₂O₃ catalyst (5% by mass of Pt) was modified with the following compounds:
N-acetylphenylalanine
N—Ac—S-phenyl-L-cysteine

The catalysts were suspended in 3 ml of the solvent. Thereafter, 1 ml of the solution of the modifier in the solvent was added and the mixture stirred at room temperature for 30 min. The chemical conversion was effected at 50° C. and a partial H₂ pressure of 5 bar in acetic acid as a solvent. One reaction batch contained in each case 10 mg of the dry catalyst and 6 ml of the reaction solution with a substrate concentration of 750 mmol/l and a modifier concentration of 0.2 mmol/l.

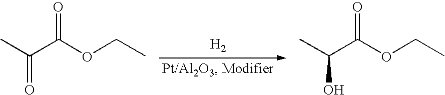

The yields and ee values are summarized in Table 5.

TABLE 5
Results of the conversion of ethyl pyruvate (40° C.,
5 bar, substrate concentration 750 mmol/l; modifier
concentration 0.2 mmol/l).

| | Reaction | | |
|---|---|---|---|
| | time | | Yield |
| Modifier | [min] | ee [%] | [%] |
| None | 600 | −0.5 | 48 |
| N-acetylphenylalanine | 600 | 8.0 | 49.0 |
| N-acetyl-S-phenyl-L-cysteine | 600 | −69.4 | 58.0 |

The inventive catalyst/modifier system exhibits the highest enantiomeric enrichment compared to the modifier-free system and to the system comprising the sulphur-free modifier under the selected reaction conditions.
i List, B. Tetrahedron Lett. 2002, 58, 5573
ii H. Lindlar, Helv. Chim. Acta 35 (1952) 446.
vi a) T. M. Tri, P. Gallezot, B. Imelik, Stud. Surf. Sci. Catal. 11 (1982) 141.
   b) C. H. Bartholomew, P. K. Agrawal, J. R. Katzer, Adv. Catal. 31 (1982) 135.
vii Sajiki, H.; Hirota, K. Tetrahedron 1998, 54, 13981.
viii H. Sajiki et al., Organic Letters, published on Web 28/06/2006
xiv K. Borszeky, T. Mallat, A. Baiker, Tetrahedron: Asym 10(24), 1999, pp. 4781-4789
xviii H.-U Glaser, B. Pugin, M. Studer in “Chiral Catalyst Immobilization and Recycling”, D. E. De Vos, I. F. J. Vankelecom, P. A. Jacobs (Eds.), Wiley-VCH, Weinheim, 2000, p. 1.
xxxix A. Tai, T. Sugimura, in “Chiral Catalyst Immobilization and Recycling”, D. E. De Vos, I. F. J. Vankelecom, P. A. Jacobs (Eds.), Wiley-VCH, Weinheim, 2000, p. 173.
xl T. Osawa, T. Harada, A. Tai, Catal. Today 37 (1997) 465.
xli H.-U Glaser, A. Indolese, A. Schnyder, H. Steiner, M. Studer, J. Mol. Catal. A: Chem. 173 (2001) 3.

1. Catalyst systems consisting of supported or unsupported transition metal catalysts whose surface has been modified with defined amounts of organic modifiers, characterized in that the modifier has a sulphur-containing functionality (G₀).

2. Catalyst systems according to claim 1, wherein the modifier has at least one further functional group (G₁) with Brønsted-basic, Brønsted-acidic, Lewis-basic or Lewis-acidic properties.

3. Catalyst systems according to claim 1, wherein the modifier has a spacer (Sp) between the sulphur-containing functionality (G₀) and the Brønsted-basic, Brønsted-acidic, Lewis-basic or Lewis-acidic functionality (G₁).

4. Catalyst systems according to claim 1, wherein the unsupported catalyst or the supported catalyst comprises one or more catalytically active components, where these components may be compounds of the elements of transition group I, II, VII and VIII of the Periodic Table and preferably compounds of the elements Pt, Pd, Rh, Ru, Re, Ir, Au, Ag, Ni, Co, Cu and Fe.

5. Use of the catalyst systems according to claim 1 for catalysis of the following reaction classes:
chemo-, stereo-, diastereo- and/or enantioselective hydrogenation of substrates which have at least one functional group or a plurality of functional groups from the group of:
one or more carbonyl groups,
one or more C═C double bonds,
one or more aromatic and/or heteroaromatic groups,
one or more nitro groups,
one or more nitrile groups,
one or more imine groups,
one or more hydroxylamine groups,
one or more alkyne groups.

6. Use of the catalyst systems according to claim 1 for the chemo-, stereo-, diastereo- or enantioselective reductive alkylation of primary or secondary amines.

7. Use of the catalyst systems according to claim 1 for the chemo-, stereo-, diastereo- or enantioselective reductive amination of aldehydes or ketones with ammonium salts or amines.
